(19) United States
(12) Patent Application Publication
(10) Pub. No.: US 2009/0215782 A1
(43) Pub. Date: Aug. 27, 2009

(54) Use of PDE-5 Inhibitors for Endothelial Repair of Tissues Impaired by Trauma or Disease

(76) Inventor: Carlo Foresta, Padova (IT)

Correspondence Address: DICKSTEIN SHAPIRO LLP, 1825 EYE STREET NW, Washington, DC, 20006-5403, US

(21) Appl. No.: 11/918,248

(22) PCT Filed: Apr. 18, 2005
(86) PCT No.: PCT/EP2005/051698

§ 371 Date: Feb. 4, 2009

(51) Int. Cl. A61K 31/53; C07D 487/02; C07D 241/36; A61K 31/4985; A61P 9/00; A61P 25/00

(52) U.S. Cl. 514/243; 544/184; 544/343; 514/250

(57) ABSTRACT

The invention relates generally to neovascularization and endothelial repair of tissues impaired by trauma or disease. Particularly, the invention relates to the function of endothelial progenitor cells in neovascularization and endothelial repair. More particularly, the invention relates to methods and pharmaceutical compositions for the treatment of tissues impaired by trauma or disease. In particular, the present invention relates to the use of a type V phosphodiesterase inhibitor in the preparation of a medicament for the treatment of a condition which is susceptible to the treatment with circulating EPCs wherein the treatment is effected by the proliferation of endothelial progenitor cells.

### TECHNICAL FIELD

The invention relates generally to neovascularization and endothelial repair of tissues impaired by trauma or disease. Particularly, the invention relates to the function of endothelial progenitor cells in neovascularization and endothelial repair. More particularly, the invention relates to methods and pharmaceutical compositions for the treatment of tissues impaired by trauma or disease.

### BACKGROUND

The endothelium is the largest organ in the body. Endothelial cells (ECs) interposed between blood and tissue, participate in numerous functions of vascular physiology. They are also key determinants of health and disease in blood vessels and play a major role in arterial disease.

The observation of ECs on the lining of a ventricular assist device, led to the discovery that endothelial cells were deposited on the lining from circulating blood (Frazier O., Baldwin R., Eskin S., Duncan J. Immunochemical identification of human endothelial cells on the lining of a ventricular assist device. Tex. Heart Inst. J., 20: 78-82, 1993). Putative progenitor ECs were isolated from peripheral blood and were shown to differentiate into ECs in vitro. In animal models of ischemia, the putative endothelial cell progenitors (EPCs) were shown to be incorporated into sites of neovascularization (Asahara T., Murohara T., Sullivan A., Silver M., Van der Zee R., Li T., Witzenbichler B., Scatteman G. Isolation of putative progenitor endothelial cells for angiogenesis. Science (Wash. DC), 275: 964-967, 1997). The role of bone marrow derived endothelial cells in promoting endothelial monolayer formation in vivo was also previously reported (Shi Q., Raffi S., Wu M., Wijelath E. S., Yu C., Ishida A., Fujita J., Kothari S., Mohle R., Sauvage L. R., Moore M. A., Storb R. F., Hammond W. P. Evidence for circulating bone marrow-derived endothelial cells. Blood, 92: 362-367, 1998). These studies indicate the existence of circulating EPCs that arise outside the sites of vascularization. Thus, EPCs from haematopoietic stem cells in bone marrow migrate into peripheral circulation, home to sites of neovascularization and differentiate into mature endothelial cells. In this way, EPCs contribute to neovascularization and to ongoing endothelial repair (Dimmeler S., Zeiher A M. Vascular Repair by circulating endothelial progenitor cells: the missing link in atherosclerosis? J Mol Med. 85, 671-677, 2004).

One crucial event for mobilization of EPCs from the bone marrow is the expression of matrix metalloproteinase 9 (MMP-9) by the hematopoietic and stromal cell compartments of the bone marrow. In turn, MMP-9 releases soluble c-Kit ligand (sKitL), which induces survival, mobilization and proliferation in the stem and progenitor cell compartments. This process allows the transfer of EPCs from the quiescent to the proliferative niche. A recent study on mice suggested the essential role of endothelial nitric oxide synthase (eNOS), which produces nitric oxide (NO) from L-arginine, for the functional activity of hematopoietic stem cells and EPCs. It has also been demonstrated that NO acts primarily in a paracrine manner to induce an increase in the number of circulating. EPCs and that a lack of eNOS induces defective hematopoietic recovery and EPC mobilization (Aicher A., Heeschen C., Dimmeler S. The role of NOS3 in stem cell mobilization. Trends Mol Med. 10, 421-425, 2004. Aicher A., Hescheen C., Mildner-Rihm C, Urbich et al. Essential role of endothelial nitric oxide synthase for mobilization of stem and progenitor cells. Nat Med. 9, 1370-1376, 2003). Nitric oxide acts by increasing the activity of guanylyl cyclases which increases the production of cyclic guanosine monophosphate (cGMP).

Approaches to neovascularize or repair damaged endothelial tissue affected by trauma or disease are of great importance. Increasing the number of EPCs at the site of trauma or disease will promote neovascularization and endothelial repair.

A technique for re-endothelializing an artery whose endothelial layer has been damaged by balloon angioplasty is described in WO0306357. The technique comprises, in one embodiment, introducing into the bloodstream of a patient, prior to performing the angioplasty, a quantity of a bispecific antibody, the bispecific antibody having a first antigen binding site directed against a surface marker common to both EPCs and endothelial cells (ECs) and having a second antigen binding site directed against a subendothelial epitope.

In U.S. Pat. No. 5,980,887, the use of EC progenitors in a method for the regulation of angiogenesis in a selected patient and in specific locations, was described. Formation of new blood vessels is induced by administering to a patient an effective amount of an isolated endothelial progenitor cell.

An increase of EPCs at the site of trauma or disease may be achieved by increasing the number of circulating EPCs. Several studies have described protocols to isolate and expand the population of circulating EPCs from blood (Asahara T., Murohara T., Sullivan A., Silver M., Van der Zee R., Li T., Witzenbichler B., Scatteman G. Isolation of putative progenitor endothelial cells for angiogenesis. Science (Wash. DC), 275: 964-967, 1997. Shi Q., Raffi S., Wu M., Wijelath E. S., Yu C., Ishida A., Fujita J., Kothari S., Mohle R., Sauvage L. R., Moore M. A., Storb R. F., Hammond W. P. Evidence for circulating bone marrow-derived endothelial cells. Blood, 92: 362-367, 1998). In different animal models of neovascularization, EPCs were shown to participate and enhance the formation of new blood vessels, and consequently increase tissue salvage (Asahara, T., Masuda, H., Takahashi, T., Kalka, C., Pastore, C., Silver, M., Kearne, M., Magner, M. and Isner, J. M. Bone marrow origin of endothelial progenitor cells responsible for postnatal vasculogenesis in physiological and pathological neovascularization. Circ. Res. 85, 221-228, 1999a. Kawamoto, A., Gwon, H. C., Iwaguro, H., Yamaguchi, J. I., Uchida, S., Masuda, H., Silver, M., Ma, H., Kearney, M., Isner, J. M. and Asahara, T. Therapeutic potential of ex vivo expanded endothelial progenitor cells for myocardial ischemia. Circulation 103,634-637, 2001). However, EPCs represent a small proportion (0.1-0.5%) of circulating blood cells, and their expansion ex-vivo after harvest takes a considerable amount of time (Sandrine Marchetti, Clotilde Gimond, Kristiina Iljin, Christine Bourcier, Kari Alitalo, Jacques Pouyssegur and Gilles Pages. Endothelial cells genetically selected from differentiating mouse embryonic stem cells incorporate at sites of neovascularization in vivo. Journal of Cell Science 115, 2075-2085, 2002).

WO0194420 describes a method of stimulating vasculogenesis of a damaged tissue in a subject comprising: (a) removing stem cells from a location in a patient; (b) recovering endothelial progenitor cells in the stem cells; (c) introducing the endothelial progenitor cells from step (b) into a different location in the patient such that the precursors migrate to and stimulate revascularization of the tissue. The endothelial progenitor cells may be expanded before introduction into the subject.

WO02089727 describes the use of C17 protein to stimulate the growth of endothelial and hematopoietic cells expressing the CD34 marker. It was suggested that C17 may be used as a tool for stimulating angiogenesis and neovascularization in wound healing and other vascular deficient disorders.

The problem underlying the invention is to provide a method of treating a patient's tissue, of a disorder, which is susceptible to endothelial progenitor cell action.

This problem is solved by the use of type V phosphodiesterase inhibitor(s) as depicted in the appended claims.

### SUMMARY OF THE INVENTION

The present invention is based on the surprising discovery that inhibitors of the type V phosphodiesterase are able to induce endothelial progenitor cell proliferation and to increase circulating EPCs in blood vessels.

An embodiment in accordance with the present invention provides a use of a type V phosphodiesterase inhibitor in the preparation of a medicament for the therapeutic and/or prophylactic treatment of a condition susceptible to treatment with circulating EPCs

Non limitative examples of preferred conditions to be treated are those arising from transplant of organs, surgery, endothelial diseases, cerebrovascular diseases, pathologies which induce decrease in circulating endothelial progenitor cells, vascular lesion, arteriovenous fistula or mechanical damage of the vascular tissue.

Preferably, the therapeutic and/or prophylactic treatment of the invention is effected by the proliferation of endothelial progenitor cells.

The medicament is, preferably, administered orally to a subject by a pharmaceutical vehicle preferably selected from the group consisting of tablets, capsules, powders and ingestible liquid.

In another embodiment, the present invention provides a method for the treatment of a condition susceptible to treatment with circulating EPCs comprising administering to the patient a therapeutically effective amount of a type V phosphodiesterase inhibitor for promoting endothelial progenitor cell proliferation so as to thereby treat the said condition.

Preferably, the amount of type V phosphodiesterase is administered at a dosage that is able to elicit the release of EPCs from bone marrow and to increase the number of circulating EPCs.

More preferably, the amount of type V phosphodiesterase inhibitor ranges from dosage from 0.14 mg/kg body weight to 0.28 mg/kg body weight every three days for a period of 6 months.

Preferably, the type V phosphodiesterase inhibitor is contained in a pharmaceutical composition.

More preferably, the type V phosphodiesterase inhibitor is selected from the group consisting of Vardenafil or Tadalafil.

Preferably, the type V phosphodiesterase inhibitor is administered to a human subject.

### BRIEF DESCRIPTION OF THE DRAWINGS

FIG. 1 depicts the gate strategy of 500,000 mononuclear cells analysed from flow cytometry.

FIG. 2 depicts the flow cytometry profile of CD34 (X axis) and AC133 (Y axis) expression from a representative untreated subjects at baseline. The percentage of positive cells is indicated in each panel.

FIG. 3 depicts the flow cytometry profile of CD34 (X axis) and AC133 (Y axis) expression from a representative subjects at four hours after administration of Vardenafil 20 mg. The percentage of positive cells is indicated in each panel.

FIG. 4 depicts the concentration of EPCs (EPC/mL) in each of the subjects at baseline and at two hours and at fours after the administration of Vardenafil 20 mg. Continuous and dashed lines indicate the mean value and the 95% confidence levels respectively.

FIG. 5 depicts the concentration of EPCs (EPC/mL) in each of the subjects at fours after the administration of Vardenafil 20 mg. Continuous and dashed lines indicate the mean value and the 95% confidence levels respectively.

### DETAILED DESCRIPTION

Before describing the present invention in detail it is to be understood that this invention is not limited to particular drugs or drug delivery systems. It is also to be understood that the terminology used herein is for the purpose of describing particular embodiments only, and is not intended to be limiting.

While this invention is described generally with reference to human subjects, veterinary applications are contemplated within the scope of this invention.

It must be noted that as used in this specification and the appended claims, the singular forms “a,” “an” and “the” include plural referents unless the context clearly dictates otherwise. Thus, for example, reference to “an active agent” or “a pharmacologically active agent” includes a single active agent as well a two or more different active agents in combination, reference to “a carrier” includes mixtures of two or more carriers as well as a single carrier, and the like.

### DEFINITIONS

In describing and claiming the present invention, the following terminology will be used in accordance with the definitions set out below:

“AC133” as used herein refers to a 120 kDa five transmembrane glycoprotein (5-TM) expressed on primitive cell populations, such as CD34 haematopoietic stem and progenitor cells, neural and endothelial progenitor cells and other primitive cells such as retina and retinoblastoma and developing epithelium. AC133 is expressed on haemagioblasts and developing endothelium, in addition to haematopoietic stem cells and neural stem cells.

“CD34” as used herein refers to a surface antigen expressed by haematopoietic precursors, capillary endothelial cells and embryonic fibroblasts. CD34 is a sialomucin, type I transmembrane protein and has certain functions: a CD62L counter-receptor, adhesion, stem cell marker.

“Active agent,” “drug” and “pharmacologically active agent” are used interchangeably herein to refer to a chemical material or compound that induces a desired effect. The primary active agents herein are type V phosphodiesterase inhibitors, although combination therapy wherein a type V phosphodiesterase inhibitor is administered with one or more additional active agents is also within the scope of the present invention. Included are derivatives and analogs of those compounds or classes of compounds specifically mentioned which also induce the desired effect.

“Blood vessels” as used herein refers to the vessels through which blood circulates. Blood vessels include arteries, veins and capillaries including capillaries of the microvascular circulation. They are elastic tubular channels lined with endothelium.

“Carriers” or “vehicles” as used herein refer to carrier materials suitable for administration of the active agent or drug. Carriers and vehicles useful herein include any such materials known in the art which is nontoxic and does not interact with other components of the composition in a deleterious manner.

“Circulating endothelial progenitor cell” as used herein is intended to mean an endothelial progenitor cell found circulating in the vascular system.

“Differentiation” as used herein refers to the process by which cells become more specialized to perform particular biological functions.

“Disorder” or “Dysfunction” are used interchangeably and include any impaired or abnormal functioning of a tissue, organ or system due to a trauma or disease or any disturbances/disruptions of the regular or normal functions of the tissue, organ or system.

“Endothelial progenitor cell” as used herein refers to a cell that can give rise to a differentiated endothelial cell. In one specific, non-limiting example, endothelial progenitor cells express a number of endothelial specific markers including receptors for vascular endothelial growth factor (VEGFR-2), CD31, Tie-2 and VE-Cadherin (Asahara T., Murohara T., Sullivan A., Silver M., Van der Zee R., Li T., Witzenbichler B., Scatteman G. Isolation of putative progenitor endothelial cells for angiogenesis. Science (Wash. DC), 275: 964-967, 1997).

“Effective” or “therapeutically effective” amount/dose of a drug or pharmacologically active agent is meant a nontoxic but sufficient amount/dose of the drug or agent to provide the desired effect, i.e., increase in the number of EPCs, neovascularization etc.

“Neovascularization” as used herein is intended to mean the development of new blood vessels from cells such as the bone marrow derived endothelial progenitor cells.

“Pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained.

“Pharmacologically active” (or simply “active”) as in a “pharmacologically active” derivative or metabolite, refers to a derivative or metabolite having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well, i.e., therapeutically effective for the treatment of trauma or disease of vascular tissue.

“Parenteral administration” used herein may be performed by subcutaneous, intramuscular or intravenous injection by means of a syringe, optionally a pen-like syringe. The formulation for parenteral administration may be in an aqueous or non-aqueous sterile injectable solutions. Such solutions may contain anti-oxidants, buffers, bacteriostats and solutes which render the formulation isotonic with the blood of the intended recipient. Alternatively, the formulation for parenteral administration may be presented as an aqueous or non-aqueous sterile suspension which may include suspending agents and thickening agents.

“Progenitor cell” as used herein refers to cells that produces progeny in a defined cell lineage. Accordingly, an endothelial progenitor cell produces cells of the endothelial lineage.

“Oral administration” is used herein to mean administration of a active agent, drug or pharmacologically active agent” in a pharmaceutical vehicle convenient for that administrative route. Thus, for example, the medicament may be presented as tablets, capsules, ingestible liquid or a powder preparation. Such formulations can include pharmaceutically acceptable carriers known to those skilled in the art. Formulations suitable for oral administration further include lozenges, pastilles, aerosols and mouthwashes.

“Transdermal” delivery, as used herein is intended to include both transdermal (or “percutaneous”) and transmucosal administration, i.e., delivery by passage of a drug through the skin or mucosal tissue and into the bloodstream.

“Transmucosal” drug delivery is meant administration of a drug to the mucosal surface of an individual so that the drug passes through the mucosal tissue and into the individual's blood stream. Transmucosal drug delivery may be “buccal” or “transbuccal,” referring to delivery of a drug by passage through an individual's buccal mucosa and into the bloodstream. Another form of transmucosal drug delivery herein is “sublingual” drug delivery, which refers to delivery of a drug by passage of a drug through an individual's sublingual mucosa and into the bloodstream.

“Transplant” as used herein includes a llograft and autograft tissue or bone transplant and allograft organ, tissue and bone transplant. Transplant of tissue or organ is intended to include grafting of skin or any other tissue or organ.

“Treating” and “treatment” as used herein refer to a reduction in severity and/or frequency of symptoms, elimination of symptoms and/or underlying cause, prevention of the occurrence of symptoms and/or their underlying cause and improvement or remediation of damage.

“Topical administration” is used in its conventional sense to mean delivery of a topical drug or pharmacologically active agent to the skin or mucosa.

“Type V phosphodiesterase inhibitor” refers to an agent that reduces (e.g. selectively reduces) or eliminates the activity of a type V phosphodiesterase. Unless otherwise indicated, the term “type V phosphodiesterase inhibitor” is intended to include type V phosphodiesterase inhibitors per se as well as salts, esters, amides, prodrugs, active metabolites and other derivatives thereof, it being understood that any salts, esters, amides, prodrugs, active metabolites or other derivatives are pharmaceutically acceptable as well as pharmacologically active.

“Vascular tissue” as used herein refers to tissue consisting of, or containing, vessels as an essential part of a structure. Vascular tissue operates by means of, or is made up of an arrangement of, vessels such as blood vessels. Vascular tissue includes the arteries, veins, capillaries, lacteals, microvasculature, endothelium etc. In one embodiment, vascular tissue is a blood vessel, or a portion thereof. In another embodiment, vascular tissue includes a highly vascularized organ (e.g. the lung).

“Vascular system” as used herein refers to the system of channels for the conveyance of a body fluid (as blood of an animal). The vascular system is comprised of blood vessels.

### Disorders Susceptible to Treatment by EPCs

EPCs from haematopoietic stem cells in bone marrow migrate into peripheral circulation and migrate to sites of neovascularization or endothelial repair and differentiate into mature endothelial cells. For the purposes of this invention endothelial repair includes the regeneration of tissues. Increasing the number of EPCs at the site in need thereof, by increasing the number of circulating EPCs, will promote neovascularization and endothelial repair at this site.

The present invention provides a method of treatment or prophylaxis of disorders which are susceptible to the action of EPCs by the administration of a type V phosphodiesterase inhibitor.

According to an embodiment of the present invention, the administration of a PDE-5 inhibitor results in the proliferation of EPCs in the bone marrow or in the differentiation of circulating haematopoietic progenitor cells into EPCs. The number of circulating EPCs will increase in a corresponding fashion. Accordingly, there will also be an increase in the number of EPCs migrating to the site to be treated, differentiating into endothelial cells and subsequently incorporation into the sites of tissue damage.

The conditions susceptible to the treatment according to the present invention are those which can occur as a result of a trauma or disease of the tissue. Trauma to the tissue can be caused by cuts, lacerations or any agent, force, or mechanism that causes damage to the tissue. Non-limiting examples are the formation of lesions in the tissue, such as arteriovenous fistula. Disease refers to a condition of a tissue or a organ or parts thereof that impairs normal functioning. Preferred diseases are those which are susceptible to the action of circulating EPCs. Non-limiting examples are, diseases which result in the need of neovascularization, aneurysms, angiodysplasia. In particular, diseases of the endothelium (Non-limiting examples: blood vessel wall bleeding, vascular ulcers) and cerebrovascular diseases (Non-limiting examples intracerebral haemorrhage, subarachnoid haemorrhage).

In accordance with an embodiment, PDE-5 inhibitor is administered in the treatment of a trauma or disease susceptible to the action of circulating EPCs.

In another embodiment, PDE-5 inhibitor is used in the treatment of subjects having pathologies which induce a decrease of circulating EPCs. The PDE-5 inhibitor serves to counter the effects of the pathology by increasing the number of circulating EPCs.

Stimulation of neovascularization and/or endothelia 1 repair can aid in vascularizing of skin grafts.

Further, neovascularization and/or endothelial repair will also assist in the treatment of subjects that have undergone transplants of organs, body parts and/or tissue. Major bleeding after a transplant is not uncommon. This is often a result of constant oozing from the raw operated surfaces or leakage from sutures or staples. Constant bleeding will reduce the amount of blood supplied to the transplanted organ, tissue or body part. Thus, an increase in the number of circulating EPCs for the stimulation of neovascularization and/or endothelial repair of vascular tissue in sites of interest will increase the likelihood of long-term cell and organ viability in a recipient. To increase the number of circulating EPCs, the PDE-5 may be administered prior to and/or after the transplant procedure, preferably after the transplant procedure.

It is envisaged that such an approach may be extended to a subject that has undergone surgery of any nature.

Thus in yet another embodiment of the invention, a PDE-5 inhibitor is administered in the treatment of a subject that has undergone surgery or transplant of an organ, tissue or body part.

Specific groups of subjects may be predisposed to a specific disorder. Augmenting the resident population of EPCs represents an effective means to minimize damage caused by an occurrence of the disorder susceptible of such treatment. Such an approach can be achieved by administering the PDE-5 inhibitor to a subject who is at risk of a disorder but has yet not been subjected to a trauma or a disease which is susceptible to treatment by the action of EPCs. For example the approach may address the issue of endothelial dysfunction or depletion that may compromise strategies of therapeutic neovascularization in older and/or diabetic, and/or hypercholesterolemic subjects.

The treatment of conditions susceptible to treatment by the action of EPCs by the administration of PDE-5 inhibitors applies generally to all mammals, preferably to humans.

The conditions in need of treatment by circulating EPCs according to the present invention can generally be recognised by the skilled man according to his common general knowledge. Those conditions that are characterized by a decrease of the number of circulating EPCs can easily be detected by counting the number of EPCs in the blood stream according to the method described herein below.

### Type V Phosphodiesterase Inhibitors

The phosphodiesterases have been classified into seven major families, types I-VII, based on amino acid or DNA sequences. As indicated by their name, phosphodiesterase inhibitors reduce or block the activity of phosphodiesterases. Phosphodiesterases are a class of intracellular enzymes involved in the metabolism of the second messenger nucleotides, cyclic adenosine monophosphate (cAMP), and cGMP. The members of the family vary in their tissue, cellular and subcellular distribution, as well as their links to cAMP and cGMP pathways. For example, the corpora cavernosa contains: type III phosphodiesterases, which are cAMP-specific cGMP inhibitable; type IV phosphodiesterases, the high affinity, high-specificity cAMP-specific form; and type V phosphodiesterases, one of the cGMP-specific forms. Inhibitors specific for each of these phosphodiesterase forms are known. The PDE-5 is the predominant isoenzyme of the PDE family in the corpora cavernosa of the penis, but its expression has been demonstrated in other organs such as smooth muscle, skeletal muscle, brain and kidney. PDE-5, which reduce or block the activity of cGMP specific phosphodiesterases, block the breakdown of cGMP.

The use of phosphodiesterase inhibitors for the treatment and prevention of diseases induced by the increased metabolism of cyclic guanosine 3′,5′-mono-phosphate (cGMP), such as hypertension, pulmonary hypertension, congestive heart failure, renal failure, myocardial infarction, stable, unstable and variant (Prinzmetal) angina, atherosclerosis, cardiac edema, renal insufficiency, nephrotic edema, hepatic edema, stroke, asthma, bronchitis, chronic obstructive pulmonary disease (COPD), cystic fibrosis, dementia, immunodeficiency, premature labor, dysmenorrhoea, benign prostatic hyperplasia (BPH), bladder outlet obstruction, incontinence, allergic rhinitis, and glucoma, and diseases characterized by disorders of gut motility, such as irritable bowel syndrome (IBS) have been previously described.

Use of selective type V phosphodiesterase (“PDE-5”) inhibitors in the treatment of erectile dysfunction is well documented as PDE-5 phosphodiesterases are held to be responsible for penile detumescence.

A recent mice model suggested an effect of PDE-5 inhibitors not only in the erectile endothelium of the corpora cavernosa, but also in other organs. It has been demonstrated that a PDE-5 inhibitor, DA-8159, induces haematological and bone marrow changes in mice suggesting a role of this class of drugs in these tissues (Shim H. J., Kim Y. C., Jang J. M., et al. Subacute toxicities and toxicokinetics of DA-8159, a new phosphodiesterase type V inhibitor, after single and 4 week repeated oral administration in rats. Biopharm Drug Dispos. 24, 409-418, 2003). Chronic administration of PDE-5 inhibitor, DA-8159, induced haematological and bone marrow modifications with increase in lymphomonocytic component count and in bone marrow density, as indicated by a decrease in adipose tissue percentage. However, the direct effect on EPCs is not known, even if EPCs are a subset of the lymphomonocyte component of the peripheral circulation.

Examples of type V phosphodiesterase inhibitors which can be used in conjunction with this invention include, but are not limited to, vardenafil, tadalafil, Zaprinast®, MY5445, dipyridamole, and Sildenafil®. Other type V phosphodiesterase inhibitors include pyrazolopyrimidinones. Examples of these inhibitor compounds include, but are not limited to 5-(2-ethoxy-5-morpholinoacetylphenyl)-1-methyl-3-n-propyl-1,6-dihydro-7H-pyrazolo[4,3-d]pyrimidin-7-one,5-(5-morpholinoacetyl-2-n-propoxyphenyl)-1-methyl-3-n-propyl-1,6-dihydro-7-H-pyrazolo[4,3-d]pyrimidin-7-one,5-[2-ethoxy-5-(4-methyl-1-piperazinylsulfonyl)-phenyl]1-methyl-3-n-propyl-1,6-dihydro-7H-pyrazolo[4,3-d]pyrimidin-7-one,5-[2-allyloxy-5-(4-methyl-1-piperazinylsulfonyl)-phenyl]-1-methyl-3-n-propyl-1,6-dihydro-7H-pyrazolo[4,3-d]pyrimidin-7-one,5-[2-ethoxy-5-[4-(2-propyl)-1-piperazinylsulfonyl)-phenyl]-1-methyl-3-n-propyl-1,6-dihydro-7H-pyrazolo[4,3-d]pyrimidin-7-one,5-[2-ethoxy-5-[4-(2-hydroxyethyl)-1-piperazinylsulfonyl)phenyl]-1-methyl-3-n-propyl-1,6-dihydro-7H-pyrazolo[4,3-d]pyrimidin-7-one,5-[5-[4-(2-hydroxyethyl)-1-piperazinylsulfonyl]-2-n-propoxyphenyl]-1-methyl-3-n-propyl-1,6-dihydro-7H-pyrazolo[4,3-d]pyrimidin-7-one,5-[2-ethoxy-5-(4-methyl-1-piperazinylcarbonyl)phenyl]-1-methyl-3-n-propyl-1,6-dihydro-7H-pyrazolo[4,3-d]pyrimidin-7-one, and 5-[2-ethoxy-5-(1-methyl-2-imidazolyl)phenyl]-1-methyl-3-n-propyl-1,6-dihydro-7H-pyrazolo[4,3-d]pyrimidin-7-one. Still other type V phosphodiesterase inhibitors useful in conjunction with the present invention include: IC-351 (ICOS); 4-bromo-5-(pyridylmethylamino)-6-[3-(4-chlorophenyl)propoxy]-3(2H)pyridazinone; 1-[4-[(1,3-benzodioxol-5-ylmethyl)amiono]-6-chloro-2-quinazolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]imidazo[2,1-b]purin-4-one; 3-acetyl-1-(2-chlorobenzyl)-2-propylindole-6-carboxylate; 4-bromo-5-(3-pyridylmethylamino)-6-(3-(4-chlorophenyl)propoxy)-3-(2H)pyrid azinone; 1-methyl-5-(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); and Sch-51866.

Other type V phosphodiesterase inhibitors include, but are not limited to DMPPO (Eddahibi (1988) Br. J. Pharmacol., 125(4): 681-688), and 1-arylnaphthalene lignan series, including 1-(3-bromo-4,5-dimethoxyphenyl)-5-chloro-3-[4-(2-hydroxyethyl)-1-piperazinylcarbonyl]-2-(methoxycarbonyl)naphthalene hydrochloride (27q) (Ukita (1999) J. Med. Chem. 42(7): 1293-1305).

In the treatment of disorders susceptible to the treatment by PDE-5 inhibitors, preferred PDE-5 inhibitors are Vardenafil and Tadalafil.

### Pharmaceutical Formulations and Modes of Administration

In order to carry out the methods of the invention, one or more PDE-5 inhibitors are administered to an individual prone to vascular disorder or having a vascular disorder. While this invention is described generally with reference to human subjects, veterinary applications are contemplated within the scope of this invention. It is envisaged, one or more type V phosphodiesterase inhibitors may be administered in conjunction with a another active agent. The PDE-5 inhibitor and the second active agent can be administered simultaneously or sequentially with either the inhibitor or the second active agent being administered first. Both the inhibitor and the second active agent can be administered by the same modality (and even in the same formulation) or they can be administered in different formulations and/or by different modalities. The PDE-5 inhibitor(s) may be administered, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

For example, acid addition salts are prepared from the free base using conventional methodology, that typically involves reaction with a suitable acid. Generally, the base form of the drug is dissolved in a polar organic solvent such as methanol or ethanol and the acid is added thereto. The resulting salt either precipitates or may be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are halide salts, such as may be prepared using hydrochloric or hydrobromic acids. Conversely, preparation of basic salts of acid moieties which may be present on a phosphodiesterase inhibitor molecule are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

Preparation of esters typically involves functionalization of hydroxyl and/or carboxyl groups which may be present within the molecular structure of the drug. The esters are typically acyl-substituted derivatives of free alcohol groups, i.e., moieties that are derived from carboxylic acids of the formula RCOOH where R is alkyl, and preferably is lower alkyl. Esters can be reconverted to the free acids, if desired, by using conventional hydrogenolysis or hydrolysis procedures.

Amides and prodrugs may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety which results in a compound that is therapeutically inactive until modified by an individual's metabolic system.

The PDE-5 inhibitor(s) and various derivatives and/or formulations thereof are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers.

The PDE-5 inhibitor(s) and various derivatives and/or formulations thereof as identified herein are useful for parenteral, topical, oral, or local administration, such as by aerosol or transdermally, for prophylactic and/or therapeutic treatment of vascular disorder. Oral administration is generally preferred.

For oral administration, a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents coloring agents, emulsifying agents and/or suspending agents, as well as such diluents such as water, ethanol, propylene glycol, glycerin and various like combinations thereof.

The pharmaceutical compositions can be administered in a variety of unit dosage forms depending upon the method of administration. Suitable unit dosage forms, include, but are not limited to powders, tablets, pills, capsules, lozenges, suppositories, etc.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s). The excipients are preferably sterile and generally free of undesirable matter. These compositions may be sterilized by conventional, well known sterilization techniques.

The concentration of active agent(s) (type V phosphodiestersase inhibitor(s)) can vary widely, and will be selected primarily based on fluid volumes, viscosities, body weight and the like in accordance with the particular mode of administration selected and the patient's needs. Concentrations, however, will typically be selected to provide dosages in accordance with the dosage recommendations provided above. It will be appreciated that such dosages may be varied to optimize a therapeutic regimen in a particular subject or group of subjects.

In therapeutic applications, the compositions of this invention are administered to a patient suffering from a condition susceptible to the action of EPCs, in an amount sufficient to cure or at least partially modify the issue of the condition and its complications (e.g diseases of the endothelium, diseases of blood vessels in the cerebrum). An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

Preferably, the PDE-5 inhibitors will be administered at a dosage that is able to elicit the release of EPCs from bone marrow and to increase the number of circulating EPCs. Typically, such a dosage will be less than the dosage needed for the treatment of erectile dysfunctions.

A preferred schedule of treatment will be administering the PDE-5 inhibitor, preferably Tadalafil, at a dosage from 0.14 mg/kg body weight to 0.28 mg/kg body weight every three days for a period of 6 months, but the dosage and period of treatment will vary with the characteristics of the PDE-5 inhibitor used.

In certain preferred embodiments, the PDE-5 inhibitor(s) is administered orally (e.g. via a tablet) or as an injectable in accordance with standard methods well known to those of skill in the art. In other preferred embodiments, the phosphodiesterase inhibitors and/or L-arginine may also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, prefer ably functions as a primary structural element of the “patch” and provides the device with much of its flexibility. The material selected for the backing layer is preferably substantially impermeable to the active agent(s) and any other materials that are present.

In the treatment of vascular disorder, it is contemplated that in certain embodiments the PDE-5 inhibitor(s) is administered locally via a patch (e.g. as described above) or other topical formulation. Other preferred formulations for topical drug delivery include, but are not limited to, ointments and creams. Ointments are semisolid preparations which are typically based on petrolatum or other petroleum derivatives. Creams containing the selected active agent, are typically viscous liquid or semiso lid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing. Oral administration is the preferred administration route according to the present invention.

The foregoing formulations and administration methods are intended to be illustrative and not limiting. It will be appreciated that, using the teaching provided herein, other suitable formulations and modes of administration can be readily devised.

### EXPERIMENTAL DETAILS

The following experiment is offered to illustrate, but not to limit the claimed invention.

### Method

10 healthy men without cardiovascular risk factors were selected for the study. The ages of the 10 men ranged from 21 to 25 years. Blood samples taken for the purposes of the study were evaluated by flow cytometry, as previously described (Scheubel R. J., Zorn H., Silber R. E., Kuss O., Morawietz H., Holtz J., Simm A. Age-dependent depression in circulating endothelial cell progenitor cells in patients undergoing coronary artery bypass grafting. J Am Coll Cardiol. 42, 2073-2080,2003).

Control samples from each subject were examined to validate the study. The samples were obtained in triplicate, on different days and at different hours of the day and examined. No significant variability was found among the subjects. This data confirmed the validity of the study.

Subjects were randomly allocated to two groups of five subjects. The first group received Vardenafil 20 mg on the first visit and a placebo one week later. The second group received the placebo on the first visit and Vardenafil 20 mg one week later. Evaluation of the number of EPCs were performed in all cases at baseline and at two hours and four hours after administration of either the placebo or Vardenafil 20 mg.

Analysis was performed on a sample of 150 μl of peripheral blood extracted from each subject. Each sample was incubated with fluorescein isothiocyanate-labelled (FITC) monoclonal antibodies against human CD34 (Becton Dickinson) and allophycocyanin (APC)-labelled monoclonal antibodies against human AC133 (Miltenyi Biotec). After incubation, the cells were lysed in NH₄Cl lysis buffer, washed twice with PBS 1% FCS and then analysed by flow cytometry.

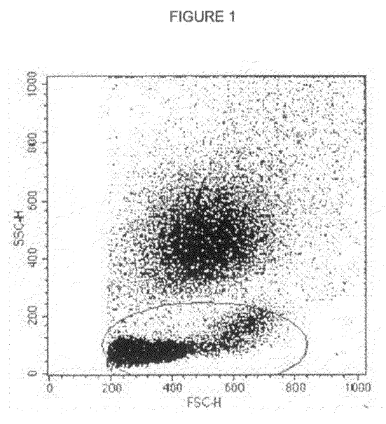

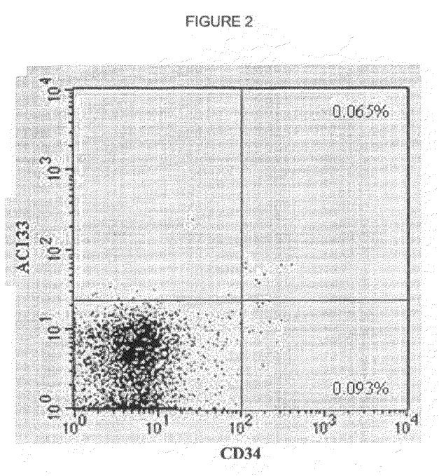

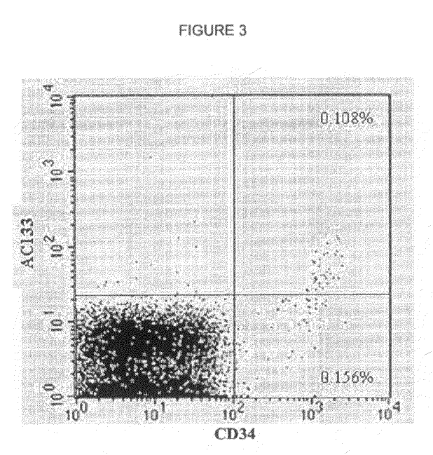

With reference to FIG. 1, forward and side scatter gates were set to include all viable mononuclear cells; into this gate 500,000 events were acquired. With reference to FIGS. 2 and 3; the upper left quadrant represent CD34 negative AC133 positive cells, the upper right quadrant represent CD34 and AC133 double positive EPCs, the lower left quadrant represent double negative CD34, AC133 cells and the lower right quadrant represent CD34 positive and AC133 negative cells. EPCs were identified by double positive events for CD34 and AC133. Specificity of staining was confirmed using equal concentration of isotype matched monoclonal antibodies. Cells were scored using a FACSCalibur analyzer (Becton Dickinson Immunocytometry Systems, San Jose, Calif., USA) and data processing using the CellQuest software programmes (Becton Dickinson Immunocytometry Systems).

The percentage of EPCs, in each subject, was calculated by dividing the number of double positive events by the number of total events. The total number of EPCs per volume, of each subject, per volume was calculated by multiplying the percentage of EPCs with the leukocyte count. The data is expressed as mean±SD. Comparisons of the total number of EPCs at baseline, and at two and four hours after the administration of either Vardenafil 20 mg or the placebo were analyzed by the Student's t-test for matched data. P values of less than 0.05 were regarded as significant.

### Results

At baseline, the concentration of EPCs in the first week was 1849.7±384.3 (95% CI: 1574.7−2124.7, range 1325-2487). At baseline, the concentration of EPCs and one week later was 1868.6±259.3. Thus, there were no significant difference in the concentration of EPCs from the first visit and one week later, among the subjects.

Administration of the placebo did not change the concentration of EPCs, neither at two hours nor at four hours.

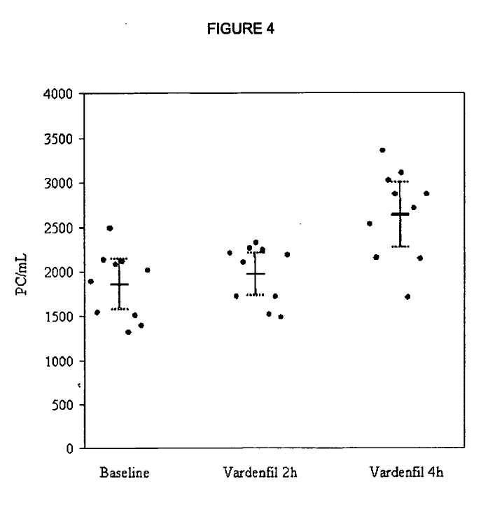

With reference to FIG. 4, two hours after administration of Vardenafil, no changes in the concentration of EPCs were observed with respect to the concentration of EPCs at baseline.

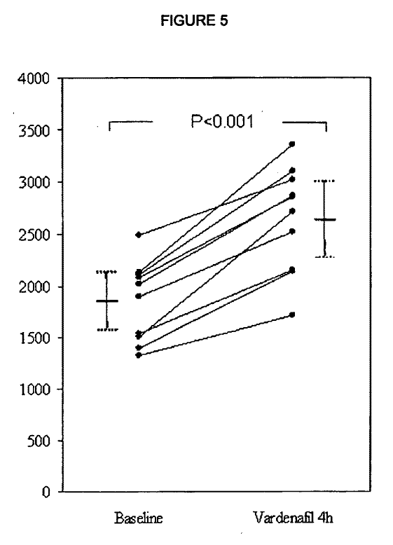

However, at four hours after the administration of Vardenafil, a significant increase in the concentration of EPCs with respect to the concentration of EPCs at baseline was observed. With reference to FIG. 5, the concentration of EPCs at four hours was 2645.4±509.5 (95% CI: 2280.9−3009.9, range 1710-3355) compared to 1849.7±384.3 at baseline. This resulted in a p value of less than 0.001. The mean increase after Vardenafil administration was 795.7±176.7 EPCs/Ml. The percentage increase was 44.1±16.5%.

### DISCUSSION

The above data show that inhibitors of type V phosphodiesterase are able to increase the circulating EPCs and thus provide a cure for the conditions which are susceptible to treatment with EPCs. Importantly, there was a surprising increase of 44.1±16.5% of circulating EPCs with respect to the baseline.

It has been previously shown that bone marrow derived EPCs incorporate into foci of neovascularization. Experiments, conducted on mice which had skin removed by punch biopsy, showed the healing of the cutaneous wounds (Asahara, T., Masuda, H., Takahashi, T., Kalka, C., Pastore, C., Silver, M., Kearne, M., Magner, M. and Isner, J. M. Bone marrow origin of endothelial progenitor cells responsible for postnatal vasculogenesis in physiological and pathological neovascularization. Circ. Res. 85, 221-228, 1999a).

The described method can also be used to analyse changes in the number of circulating EPCs. Such an analysis is useful when examining the efficacy of administering PDE-5 inhibitors in the treatment of subjects having pathologies which induce a decrease of circulating EPCs.

1. Use of a type V phosphodiesterase inhibitor in the preparation of a medicament for the treatment of a condition which is susceptible to the treatment with circulating EPCs wherein the treatment is effected by the proliferation of endothelial progenitor cells.

2. The use of a type V phosphodiesterase inhibitor as claimed in claim 1 wherein the condition is a trauma or a disease of a tissue.

3. The use of a type V phosphodiesterase inhibitor as claimed in claims 1 or 2 wherein the condition is selected from the group consisting of a transplant, surgery, endothelial disease, cerebrovascular disease, pathology which induce decrease in circulating endothelial progenitor cells, vascular lesion, arteriovenous fistula and mechanical damage of the tissue.

4. The use of a type V phosphodiesterase inhibitor as claimed in any one of claims 1 to 3 wherein the treatment is therapeutic and/or prophylactic.

5. The use of a type V phosphodiesterase inhibitor as claimed in any one of claims 1 to 4 wherein the medicament is administered to a subject in a pharmaceutical vehicle selected from the group consisting of tablets, capsules, powders and ingestible liquid.

6. The use of a type V phosphodiesterase inhibitor as claimed in any one of claims 1 to 5, wherein the type V phosphodiesterase inhibitor is administered at a dosage that is able to elicit the release of EPCs from bone marrow and to increase the number of circulating EPCs.

7. The use type V phosphodiesterase inhibitor as claimed in claim 6 wherein the dosage ranges from 0.14 mg/kg body weight to 0.28 mg/kg body weight every three days for a period of 6 months.

8. The use of a type V phosphodiesterase inhibitor as claimed in any one of claims 1 to 7 wherein the type V phosphodiesterase inhibitor is selected from the group consisting of Vardenafil or Tadalafil.

9. The use of a type V phosphodiesterase inhibitor as claimed in any one of claims 1 to 8 wherein the type V phosphodiesterase inhibitor is administered to a human subject.

10. A method for the treatment of a condition susceptible to treatment with circulating endothelial progenitor cells comprising administering a therapeutically effective amount of a type V phosphodiesterase inhibitor to a subject in need thereof.

11. The method as claimed in claim 10 wherein the condition is a trauma or a disease of a tissue.

12. The method as claimed in claims 10 or 11 wherein the condition is selected from the group consisting of a transplant, surgery, endothelial disease, cerebrovascular disease, pathology which induce decrease in circulating endothelial progenitor cell, vascular lesion, arteriovenious fistula and mechanical damage of the vascular tissue.

13. The method as claimed in any one of claims 10 to 12 wherein the treatment is therapeutic and/or prophylactic.

14. The method as claimed in any one of claims 10 to 13 wherein administering the type V phosphodiesterase inhibitor induces proliferation of endothelial progenitor cell so as to thereby treat the condition.

15. The method as claimed in any one of claims 10 to 14 wherein the type V phosphodiesterase inhibitor is contained in a pharmaceutical composition.

16. The method as claimed in any one of claims 10 to 15 wherein the type V phosphodiesterase inhibitor is administered to the subject by a pharmaceutical vehicle selected from the group consisting of tablets, capsules, powders and ingestible liquid.

17. The method as claimed any one of claims 10 to 16, wherein the type V phosphodiesterase inhibitor is administered at a dosage that is able to elicit the release of EPCs from bone marrow and to increase the number of circulating EPCs.

18. The method as claimed in claim 17 wherein the dosage ranges from 0.14 mg/kg body weight to 0.28 mg/kg body weight every three days for a period of 6 months.

19. The method as claimed in any one claims 10 to 18 wherein the type V phosphodiesterase inhibitor is selected from the group consisting of Vardenafil or Tadalafil.

20. The method as claimed in any one claims 10 to 19 wherein the type V phosphodiesterase inhibitor is administered to a human subject.
